(19) United States
(12) Patent Application Publication
(10) Pub. No.: US 2006/0088588 A1
(43) Pub. Date: Apr. 27, 2006

(54) Medication administering device and system

(76) Inventor: Lourdes Pila-Collazo, Miami, FL (US)

Correspondence Address: Davis P. Lhota;Stearns Weaver Miller, Suite 2100, 200 East Las Olas Boulevard, Fort Lauderdale, FL, 33301, US

(21) Appl. No.: 11/256,223

(22) Filed: Oct. 21, 2005

Related U.S. Application Data
(60) Provisional application No. 60/621,326, filed on Oct. 21, 2004.

(51) Int. Cl. A61K 9/48

(52) U.S. Cl. 424/451

(57) ABSTRACT

A suppository method and device for taking or administering “over-the-counter” and prescription including a capsule formed by a dissolvable material and specific quantity of a predetermined medication stored or storable in the capsule and sterilized light-resistant, moisture-proof foil peel-back packet for storage and packaging prior to use, which may be adapted for individually holding a plurality of capsules.

### CROSS REFERENCE TO RELATED APPLICATIONS

This application claims the benefit of provisional patent application Ser. No. 60/621,326 filed Oct. 21, 2004.

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyrights rights whatsoever.

### BACKGROUND OF THE INVENTION

1. Field of the Invention

This invention relates generally to medication administering device, and more particularly, to a suppository method and device for administering over-the-counter and prescription drugs to patients that cannot tolerate orally administered medication.

2. Description of the Background Art

Many people cannot tolerate oral medications because of nausea, vomiting, allergies to additives, taste or irritation. However, over-the-counter and prescription medication typically come in an orally ingested form. Consequently, they fail to take the medication or expel the medication before they receive its benefit. For instance, children often refuse to swallow medication. In fact, some adults experience a gag reflex when trying to swallow a pill. If a suppository medication administering device existed for delivery medication, it would offer an alternative to oral medications and allow people that cannot tolerate oral medications to take common medications, such as ibuprofen. The instant invention addresses this problem by providing a suppository device for administering medication as contemplated by the instant invention described herein.

### BRIEF SUMMARY OF THE INVENTION

In light of the foregoing, it is an object of the present invention to provide a non-oral medication administering device for conventional oral medication in accordance with the instant invention.

It is also an object of the instant invention to provide a medication suppository device in accordance with the instant invention.

It is another object of the instant invention to provide an alternative to oral medication in accordance with the instant invention.

In light of these and other objects, the instant invention comprises a suppository method and device for taking or administering “over-the-counter” or prescription drugs. The medication suppository device permits patients that cannot tolerate oral medications because of nausea, vomiting, allergies to additives, bad taste or irritability to receive traditional oral medications. The medication suppository device comprises a capsule formed by a dissolvable material and specific quantity of a predetermined medication stored or storable in the capsule. The capsule may be secured in a sterilized light-resistant, moisture-proof foil peel-back packet for storage and packaging prior to use. The medication suppository system comprises a plurality of capsules each containing a predetermined medication secured in packaging.

In accordance with these and other objects, which will become apparent hereinafter, the instant invention will now be described with particular reference to the accompanying drawings.

### BRIEF DESCRIPTION OF THE SEVERAL VIEWS OF THE DRAWINGS

FIG. 1 is an elevational view of the preferred embodiment of the medication administering device in accordance with the instant invention.

FIG. 2 is an elevational view of the preferred embodiment of the medication administering device in a packet in accordance with the instant invention.

FIG. 3 is an elevational view of the preferred embodiment of the packaging system for a plurality of medication administering devices in accordance with the instant invention.

### DETAILED DESCRIPTION OF THE INVENTION

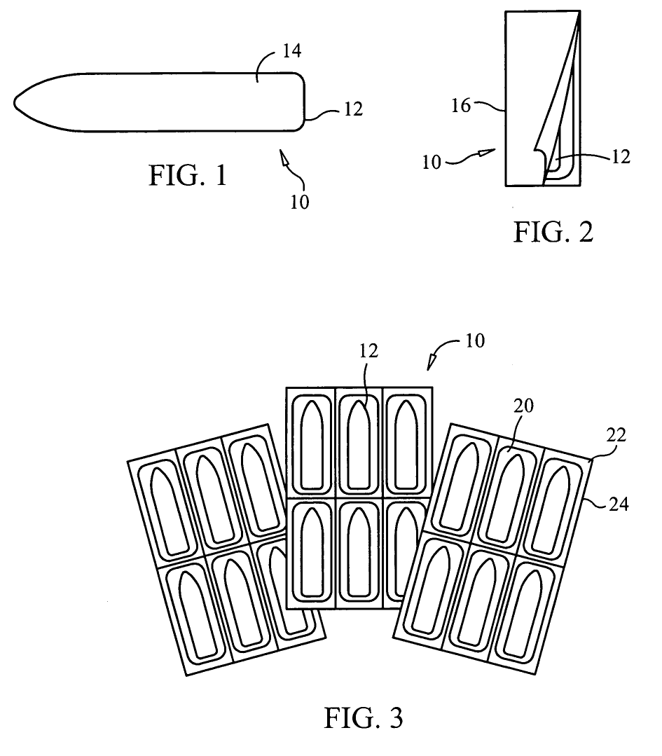

With reference to the drawings, FIGS. 1 to 3 depict the preferred embodiment of the instant invention which is generally referenced as a medication suppository device or system and, or by numeric character 10. The medication suppository device 10 of the instant invention comprises a method and device for taking or administering “over-the-counter” or prescription drugs when inserted into a person's rectum. The medication suppository device 10 permits patients that cannot tolerate oral medications because of nausea, vomiting, allergies to additives, bad taste or irritability to receive medication.

With reference to FIG. 1, the medication suppository device 10 comprises a capsule 12 formed by a dissolvable material that dissolves when inserted into a bodily passage, such as the rectum, and specific quantity of a predetermined medication 14 stored or storable in the capsule 12. The capsule 12 may be secured in a sterilized light-resistant, moisture-proof foil peel-back packet 16 for storage and packaging prior to use, as shown in FIG. 2. Referring to FIG. 3, the medication suppository system 10 comprises a plurality of capsules 12 each containing a predetermined medication 14 secured in packaging 20. The packaging 20 comprises a plurality of individual packets 22, each containing a capsule 12. The packets 22 are divided by perforated lines that facilitate convenient separation and removably secure the capsules 12 with a foil backing 24. The foil backing 24 may be punctured or peeled back to access the capsule 12.

The medication 14 in the suppository device 10 preferably comprises a predetermined quantity of Ibuprofen [(±)-2-(p-isobutylphenyl) propionic acid] or a predetermined quantity of Amoxicillin [(2S,5R,6R)-6-[(R)-(−)-2-amino-2-(p-hydroxyphenyl)acetamido]-3,3-dimethyl-7-oxo-4-thia-1-azabi cyclo[3.2.0]heptane-2-carboxylic acid trihydrate] in a matrix of Hydrogenated Vegetable Oil, Polyoxyethylene Stearate, Polysorbate 80 and other inactive ingredients. The medication 14 is placed or molded into the rectal suppository capsule 12 and may be packaged in a light-resistant, moisture-proof wrapper 16, as shown in FIG. 2, and, or in packaging 20 comprising a plurality of capsules 12.

The medication suppository device 10 may be manufactured with medication 14 having active ingredients of various strengths. The medication suppository devices 10 may be coded, such as color coded, according to the strength of its medication 14. For instance, the suppository devices 10 may be packaged in different colored wrappers or packages to identify the strength of its active ingredient(s).

The capsule 12 may be manufactured with the active ingredient compounded in a matrix of Hydrogenated Vegetable Oil, Polyoxyethylene Stearate, Polysorbate 80 and other inactive ingredients. The capsule 12 may also contain colloidal silicon dioxide, croscarmellose sodium, hydroxpropyl methylcellulose, magnesium stearate and microcrystalline cellulose to increase the firmness of the suppository.

Based on the foregoing, the medication suppository device and system 10 comprises a method and device to administer “over-the-counter” or prescription medication to patients. In one embodiment, the medication 14 in the capsule 12 includes a specific quantity of Ibuprofen [(±)-2-(p-isobutylphenyl) propionic acid]. In another embodiment, the capsule 12 may have a specific quantity of Amoxicillin [(2S,5R,6R)-6-[(R)-(−)-2-amino-2-(p-hydroxyphenyl)acetamido]-3,3-dimethyl-7-oxo-4-thia-1-azabi cyclo[3.2.0]heptane-2-carboxylic acid trihydrate]. The medication suppository device 10 may be manufactured with medication 14 having varying strengths and levels of the active ingredient(s). The inactive ingredients comprising the matrix of the medication suppository device 10 may include Hydrogenated Vegetable Oil, Polyoxyethylene Stearate, Polysorbate 80, and, or colloidal silicon dioxide, croscarmellose sodium, hydroxypropyl methylcellulose, magnesium stearate and microcrystalline cellulose to increase the firmness of the suppository device 10. The mixture of active and inactive ingredients is molded into or contained within the rectal suppository capsule 12. The capsules 12 may be individually packaged in a light-resistant, moisture-proof wrapper 16. The capsules 12 or wrappers 16 may be color coded to identify the strength or quantity of the active ingredient(s).

The medication suppository device and system 10 preferably comprises medication 14 comprising Ibuprofen. The instant invention 10 is designed for people or patients having pain and/or fever but who cannot tolerate oral medications because of nausea, vomiting, allergies to additives in liquid preparations, bad taste or irritability. The medication 14 may alternatively comprise Amoxicillin for people or patients with gram-positive and/or gram-negative bactericidal infections who cannot tolerate oral medications because of nausea, vomiting, allergies to additives in liquid preparations, bad taste, irritability or coma. When required, a capsule 12 is removed from the wrapper 15 and, or packaging 20 and fully inserted into the rectum. The medication suppository device and, or system 10 should be stored at refrigerated temperatures away from sunlight. The medication suppository device and system 10 should not be used if there is an adverse or allergic reaction to the medication 14.

The instant invention has been shown and described herein in what is considered to be the most practical and preferred embodiment. It is recognized, however, that departures may be made therefrom within the scope of the invention and that obvious structural and/or functional modifications will occur to a person skilled in the art.

What is claimed is:

1. A medication suppository device for administering medication in the device, said device comprising:
a capsule housing, said capsule housing comprising a dissolvable membrane that dissolves at a predetermined rate when inserted into a bodily passage;
a predetermined amount of medication stored in said housing; and
sterilized light-resistant, moisture-resistant foil packet storing said housing.

2. A device as recited in claim 1, further comprising:
a plurality of said packets, each said packet containing one said capsule, said packets being joined together at a plurality of points.

3. A device as recited in claim 1, wherein said packet comprises:
a foil backing.

4. A device as recited in claim 1, further comprising:
hydrogenated vegetable oil in said housing.

5. A device as recited in claim 1, further comprising:
polyoxyethylene stearate in said housing.

6. A device as recited in claim 1, further comprising:
polysorbate in said housing.

7. A device as recited in claim 1, further comprising:
polyoxyethylene stearate, polysorbate and hydrogenated vegetable oil in said housing.

8. A device as recited in claim 7, wherein said medication comprises:
ibuprofen.

9. A device as recited in claim 1, wherein said medication comprises:
ibuprofen.

10. A device as recited in claim 7, wherein said medication comprises:
amoxicillin.

11. A device as recited in claim 1, wherein said medication comprises:
amoxicillin.

12. A device as recited in claim 1, further comprising:
a code on said packet for indicating the strength of said medication in said housing.

13. A device as recited in claim 1, further comprising:
a plurality of said packets joined together at a plurality of points.

14. A device as recited in claim 1, further comprising:
colloidal silicon dioxide.

15. A device as recited in claim 1, further comprising:
croscarmellose sodium.

16. A device as recited in claim 1, further comprising:
hydroxpropyl methycellulose.

17. A device as recited in claim 1, further comprising:
magnesium stearate.

18. A device as recited in claim 1, further comprising:
microcrystalline cellulose.
